(19) United States
(12) Patent Application Publication
(10) Pub. No.: US 2022/0032164 A1
(43) Pub. Date: Feb. 3, 2022

(54) LOW-PROFILE MOUTH GUARD

(71) Applicant: Nippon Sigmax Co., LTD., Tokyo (JP)

(72) Inventors: Atsushi Adachi, Tokyo (JP); Hidenori Kurokawa, Tokyo (JP); Lance Hussey, Thousand Oaks, CA (US); Mike Schuffert, Thousand Oaks, CA (US); John Vernon, Thousand Oaks, CA (US); John Michael Elam, Thousand Oaks, CA (US); Dean Loock, Thousand Oaks, CA (US); Ravi Sawhney, Thousand Oaks, CA (US)

(73) Assignee: Nippon Sigmax Co., LTD., Tokyo (JP)

(21) Appl. No.: 17/387,969

(22) Filed: Jul. 28, 2021

Related U.S. Application Data
(60) Provisional application No. 63/057,465, filed on Jul. 28, 2020.

(51) Int. Cl. A63B 71/08; A61F 5/56; C08L 33/06; C08L 23/08; C08L 75/04; C08L 25/06; C08L 21/00

(52) U.S. Cl. CPC A63B 71/085; A61F 5/566; C08L 33/06; C08L 23/0853; C08L 2207/04; C08L 25/06; C08L 21/00; C08L 2203/02; C08L 75/04

(57) ABSTRACT

Described is a low-profile mouth guard formed of layered materials with variable hardness and different molding temperatures joined together to form a composite structure. The mouth guard is formed from a unique blend of materials including PCL (Polycaprolactate), EVA (Polyethylene Polyvinylacetate) and TPU (Thermoplastic Urethane). The unique blend allows a low-profile design that delivers both comfort and protection.

### CROSS-REFERENCE TO RELATED APPLICATIONS

This is a non-provisional application of U.S. Provisional Application No. 63/057,465, filed on Jul. 28, 2020, the entirety of which is incorporated herein by reference.

### FIELD OF INVENTION

The present invention relates to a mouth guard and, more particularly, to a low-profile mouth guard that uses a unique blend of materials to create a proprietary low-profile design that delivers both comfort and protection.

### BACKGROUND OF THE INVENTION

Mouth guards have long been known in the art. Generally, mouth guards are categorized into three different categories, a stock mouth guard, a boil type, and a malleable type. The stock mouth guards are inexpensive and bulky and suffer a major drawback in terms of their fit. The second type of mouth guard known in the prior art are boil and bite type of mouth guards. The boil type of mouth guards provide a better fit than the stock mouth guard; however, these mouth guards are generally very rigid and somewhat bulky, which decreases comfort. Improving upon flexibility, the malleable type of mouthguards are very flexible and take the shape of teeth when placed in mouth. Nevertheless, even the malleable type of mouth guards are relatively bulky and can be uncomfortable to wear.

Thus, there is a continuing need exists for a low-profile mouth guard that delivers both comfort and protection while protecting the mouth and fitting perfectly.

### SUMMARY OF THE INVENTION

The present disclosure is directed to a low-profile mouth guard. The mouth guard is formed to include an outer shell formed of a non-moldable material. The outer shell includes a first surface and a second surface opposite the first surface. An intermediate layer is formed of a first moldable material. The intermediate layer is over-molded to the second surface of the outer shell. Further, an inner layer is formed of a second moldable material. The inner layer is over-molded onto at least one of the intermediate layer and outer shell. Finally, the inner layer is transitionable from a first state to a second state, the first state being a substantially flat unmolded state and the second state being a molded state.

In another aspect, the second surface of the outer shell includes a bottom periphery, the bottom periphery being formed with a plurality of holes passing therethrough.

In yet another aspect, the inner layer includes a plurality of posts, the plurality of posts being formed through the plurality of holes.

Further, the intermediate layer is formed to cover the plurality of hole and posts formed therethrough.

Additionally, the plurality of holes are formed as countersunk holes, with the plurality of posts flaring into the countersunk holes.

In another aspect, the outer shell is made up of a material selected from a group consisting of Thermoplastic urethane, Styrene Block Polymer or rubber, Further, the inner layer is made of Polycaprolactate (PCL) material. Finally, the intermediate layer is made of polyethylene polyvinyl acetate (EVA) material.

In yet another aspect, the disclosure also includes a method for placing a mouth guard in the mouth of a user for a low profile fit. The method comprises acts of placing the mouth guard in hot water for a minimum time interval; retrieving the mouth guard from the hot water to allow the mouth guard to come to a moderate temperature; positioning a non-moldable portion of the mouth guard inside the mouth; pressing a first moldable portion of the mouth guard by applying a force towards the teeth; and folding up a second moldable portion of the mouth guard against palate of the mouth for a low profile fit.

### BRIEF DESCRIPTION OF THE DRAWINGS

The objects, features and advantages of the present invention will be apparent from the following detailed descriptions of the various aspects of the invention in conjunction with reference to the following drawings, where:

FIG. 1 illustrates a perspective view of a mouth guard in a non-molded state in accordance with a first embodiment of the present invention;

FIG. 2 illustrates an exploded view of the mouth guard in the non-molded state in accordance with the first embodiment of the present invention;

FIG. 3 illustrates a perspective view of the mouth guard in a molded state in accordance with the first embodiment of the present invention;

FIG. 4 illustrates an exploded view of the mouth guard in the molded state in accordance with the first embodiment of the present invention;

FIG. 5 illustrates a front view of the mouth guard in a non-molded state in accordance with the first embodiment of the present invention;

FIG. 6 illustrates a front view of the mouth guard in a molded state in accordance with the first embodiment of the present invention;

FIG. 7 illustrates a top view of the mouth guard in the non-molded state in accordance with the first embodiment of the present invention;

FIG. 8 illustrates an exploded view from the top of the mouth guard in the non-molded state in accordance with the first embodiment of the present invention;

FIG. 9 illustrates a top view of the mouth guard in the molded state in accordance with the first embodiment of the present invention;

FIG. 10 illustrates an exploded view from the top of the mouth guard in the molded state in accordance with the first embodiment of the present invention;

FIG. 11 illustrates a cross-sectional view of the mouth guard, taken from line A-A of FIG. 7;

FIG. 12 illustrates a cross-sectional view of the mouth guard, taken from line B-B of FIG. 9;

FIG. 13 illustrates a side view of the mouth guard in the non-molded state in accordance with the first embodiment of the present invention;

FIG. 14 illustrates a side view of the mouth guard in the molded state in accordance with the first embodiment of the present invention;

FIG. 15 illustrates a cross-sectional view of an intermediate layer and an inner layer over-molded onto an outer shell of the mouth guard in accordance with a second embodiment of the present invention;

FIG. 16 illustrates a perspective view of the mouth guard in accordance with the second embodiment of the present invention;

FIG. 17 illustrates a cross sectional view of the intermediate layer of the mouth guard in accordance with the second embodiment of the present invention;

FIG. 18 illustrates a perspective view of the intermediate layer of the mouth guard in accordance with the second embodiment of the present invention;

FIG. 19 illustrates a cross-sectional view of the intermediate layer over-molded onto the outer shell of the mouth guard in accordance with the second embodiment of the present invention;

FIG. 20 illustrates a perspective view of the intermediate layer over-molded onto the outer shell of the mouth guard in accordance with the second embodiment of the present invention;

FIG. 21 illustrates a cross-sectional view of the inner layer of the mouth guard in accordance with the second embodiment of the present invention;

FIG. 22 illustrates a perspective view of the inner layer of the mouth guard in accordance with the second embodiment of the present invention;

FIG. 23 illustrates a cross-sectional view of the outer shell of the mouth guard in accordance with the second embodiment of the present invention;

FIG. 24 illustrates a perspective view of the outer shell of the mouth guard in accordance with the second embodiment of the present invention;

FIG. 25 illustrates a cross-sectional view of an intermediate layer and inner layer over-molded onto an outer shell of the mouth guard in accordance with a third embodiment of the present invention;

FIG. 26 illustrates a perspective view of the intermediate layer and the inner layer over-molded onto the outer shell of the mouth guard in accordance with the third embodiment of the present invention;

FIG. 27 illustrates a cross-sectional view of the intermediate layer in accordance with the third embodiment of the present invention;

FIG. 28 illustrates a perspective view of the intermediate layer in accordance with the third embodiment of the present invention;

FIG. 29 illustrates a cross sectional view of the intermediate layer over-molded onto the outer shell of the mouth guard in accordance with the third embodiment of the present invention;

FIG. 30 illustrates a perspective view of the intermediate layer over-molded onto the outer shell of the mouth guard in accordance with the third embodiment of the present invention;

FIG. 31 illustrates a cross-sectional view of the inner layer in accordance with the third embodiment of the present invention;

FIG. 32 illustrates a perspective view of the inner layer in accordance with the third embodiment of the present invention;

FIG. 33 illustrates a cross-sectional view of the outer shell in accordance with the third embodiment of the present invention;

FIG. 34 illustrates a perspective view of the outer shell in accordance with the third embodiment of the present invention; and

FIG. 35 illustrates a method of placing a mouth guard in the mouth of a user in accordance with an embodiment of the present invention.

### DETAILED DESCRIPTION

The present invention relates to a mouthguard and, more particularly, to a low-profile mouthguard that uses a unique blend of materials to create a proprietary low-profile design that delivers both comfort and protection. The following description is presented to enable one of ordinary skill in the art to make and use the invention and to incorporate it in the context of particular applications. Various modifications, as well as a variety of uses in different applications will be readily apparent to those skilled in the art, and the general principles defined herein may be applied to a wide range of embodiments. Thus, the present invention is not intended to be limited to the embodiments presented, but is to be accorded the widest scope consistent with the principles and novel features disclosed herein.

In the following detailed description, numerous specific details are set forth in order to provide a more thorough understanding of the present invention. However, it will be apparent to one skilled in the art that the present invention may be practiced without necessarily being limited to these specific details. In other instances, well-known structures and devices are shown in block diagram form, rather than in detail, in order to avoid obscuring the present invention.

The reader's attention is directed to all papers and documents which are filed concurrently with this specification and which are open to public inspection with this specification, and the contents of all such papers and documents are incorporated herein by reference. All the features disclosed in this specification, (including any accompanying claims, abstract, and drawings) may be replaced by alternative features serving the same, equivalent or similar purpose, unless expressly stated otherwise. Thus, unless expressly stated otherwise, each feature disclosed is only one example of a generic series of equivalent or similar features.

Furthermore, any element in a claim that does not explicitly state “means for” performing a specified function, or “step for” performing a specific function, is not to be interpreted as a “means” or “step” clause as specified in 35 U.S.C. Section 112, Paragraph 6. In particular, the use of “step of” or “act of” in the claims herein is not intended to invoke the provisions of 35 U.S.C. 112, Paragraph 6.

Please note, if used, the labels left, right, front, back, top, bottom, forward, reverse, clockwise and counter clockwise have been used for convenience purposes only and are not intended to imply any particular fixed direction. Instead, they are used to reflect relative locations and/or directions between various portions of an object.

(1) Specific Aspects

Mouth guards have been devised to address a variety of needs For example, mouth guards are generally used to protect teeth from clenching or grinding while sleeping. Mouth guards are also often used to protect the wearer from injuries while playing sports. Finally, mouth guards are also commonly used to reduce snoring and provide relief from obstructive sleep apnea. A variety of mouth guards have been developed to address these various needs. Notably, existing mouth guards are bulky and due to their structural rigidity, can be uncomfortable to wear. The present invention improves upon the prior art by providing a mouth guard with a non-moldable layer to protect the teeth, while also including a number of thin moldable layers to provide a custom fit. Thus, through the use of the thin moldable layers, the mouth guard of the present invention is an improved, low-profile mouth guard.

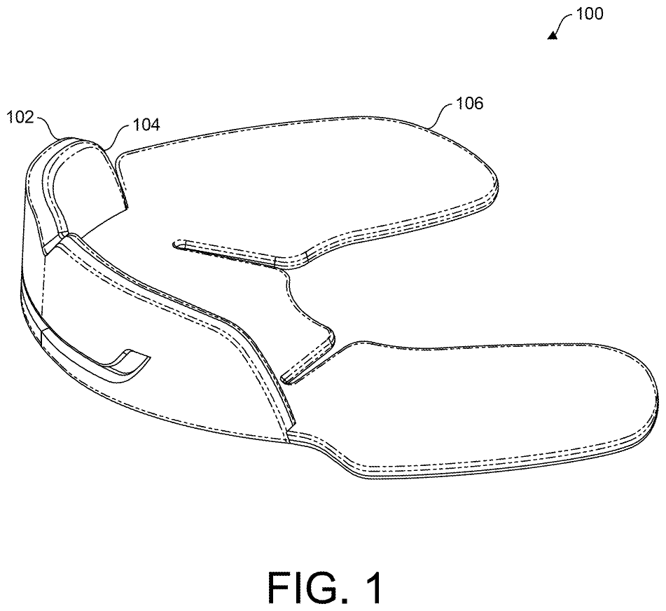

For further understanding, FIGS. 1 through 14 depict a first embodiment of the mouth guard. Specifically, FIG. 1 illustrates a perspective view of a mouth guard in a non-molded state in accordance with the first embodiment. The mouth guard is designed for two different states, a non-molded state or flat state and a molded state. The non-molded state is the flattened state that the mouth guard is presented in when purchased. As shown in FIG. 1, the mouth guard 100 is a low-profile mouth guard that uses a unique blend of materials to create a proprietary low-profile design that delivers both comfort and protection. The mouth guard 100 comprises an outer shell 102 (non-moldable layer) including a first surface and a second surface. A pair of moldable layers are adhered to the outer shell 102. The moldable layers include an intermediate layer 104 and an inner layer 106. Desirably, the pair of moldable layers are horseshoe shaped with a semicircular arc and a pair of identical sides or fins. The two pair of sides or fins helps the mouth guard fit better to the mouth and increase the ability to mold efficiently to the palate, which greatly impacts the ability to talk. All these contributing factors, including the unique blend of materials, the low profile form factor and the palate forming design provide an improvement over the prior art.

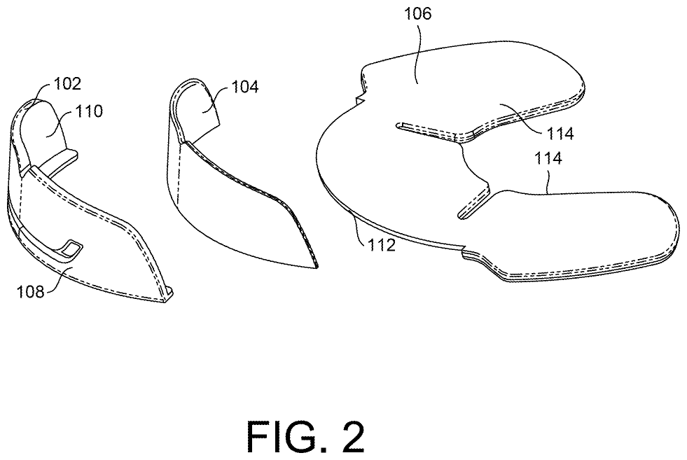

FIG. 2 illustrates an exploded view of the mouth guard in the non-molded state in accordance with the first embodiment. In one aspect, the mouth guard uses a unique blend of materials, including PCL (Polycaprolactate), EVA (Polyethylene Polyvinylacetate), and TPU (Thermoplastic Urethane), SBC (Styrene Block Polymer) or rubber to create a proprietary low profile design that delivers both comfort and protection. As noted above, the mouth guard 100 comprises of an outer shell 102 (non-moldable layer) including a first surface 108 and a second surface 110, and an intermediate layer 104. The inner layer 106 is horseshoe shaped with a semicircular arc 112 and a pair of identical sides or fins 114. Thus, the mouth guard is in the form of a layered structure having plurality of layers of varying hardness and different molding temperatures, joined together to form a composite structure. The entire structure can be segregated into three layers (as referenced above) that are fused or bonded to each other. In another aspect, the three layers can be molded together.

The inner layer 106 can be formed of a material having low shore hardness and molding temperature. For example, the inner layer 106 is made of materials, such as polycaprolactate due, to its low shear hardness and low molding temperature. Due to its low molding temperature, the inner layer 106 becomes moldable and folds up to form to the teeth. Further, the mouth guard has an intermediate layer 104 made of polyethylene polyvinyl acetate (EVA). Finally and as noted above, the mouth guard 100 includes a non-moldable layer that serves as that outer shell 102. The outer shell 102 is formed of a material having a high shore hardness and high molding temperature, selected of a material that is not moldable, even when in contact with hot water. The higher level of hardness of the outer shell 102 allows for “shut-offs” in the mold during the two over-molding steps to prevent flashing of the over-molded materials at the junctions of the materials. The outer shell 102 is made of materials selected from thermoplastic urethane (TPU) or a styrene block polymer (SBC) or rubber, while still keeping the mouth guard slim and low profile. Further, the inner layer 106 is formed of a malleable and moldable, material, a non-limiting example of which includes poly(i-caprolactone) (PCL). As illustrated, the inner layer 106 is in contact with the intermediate layer 104 and outer shell 102 forming a substrate.

The low-profile design and poly(i-caprolactone) (PCL) material allow users to talk more clearly and breath more freely. The PCL of the inner layer 106 molds to the teeth very tightly to ensure a low-profile fit, as well as helping to reduce bulk in the mouth which can affect the ability to talk and breath. The outer front guard (outer shell 102) made up of TPU (SBC, Rubber) uses a blend of materials that delivers shock absorption to increase protection, while still keeping the mouth guard slim and low profile. The outer shell 102 (TPU, SBC, Rubber etc.) does not mold under boil and bite temperature, which enables it to offer structure for better handling when molding and better protection when in use. Alternatively, the intermediate layer 104 (EVA) on the inner front guard molds under boil and bite temperatures to the teeth, which creates an enhanced fit for teeth registration and adds comfort to the user. The design also increases the moldability and fit in the mouth with the palate design, which uses PCL that molds under boil and bite temperatures and folds up against the palate of the mouth for a better, slimmer fit that increases the ability to talk and breath. The two slits in the PCL material of the inner layer 106 help the mouth guard fit better to the mouth and increase the ability to mold efficiently to the palate, which greatly impacts the ability to talk.

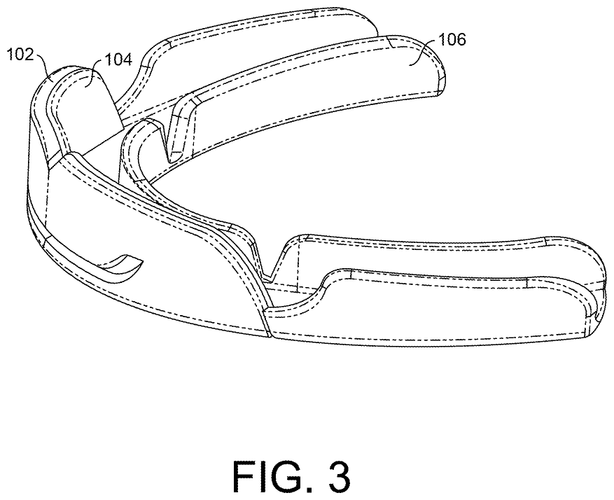

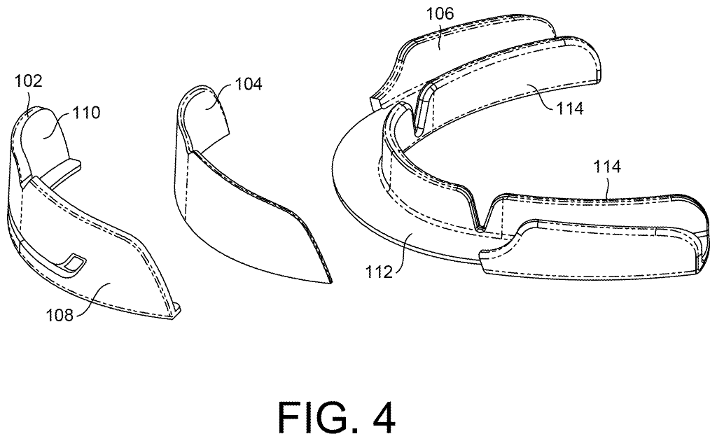

FIG. 3 illustrates a perspective view of the mouth guard in a molded state in accordance with the first embodiment, while FIG. 4 illustrates an exploded view of the mouth guard in the molded state. The objective is to provide the mouth guard designed for two different states a flat state and a molded state. As noted above, the flat state (as shown in FIG. 1) is the state of the mouth guard when it is purchased, while the second or molded state is the state in which the layers of the mouth guard get folded after coming into contact with hot water.

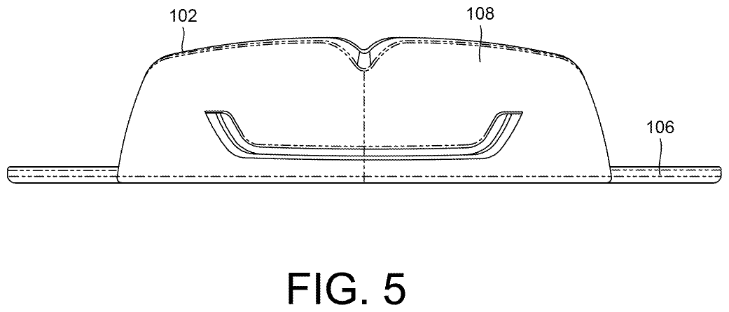

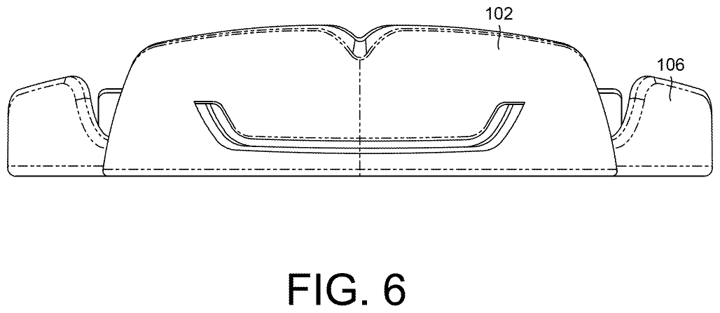

FIG. 5 illustrates a front view of the mouth guard in the non-molded state in accordance with the first embodiment. As shown, the inner layer 106 is flattened for shipping and packaging purposes. FIG. 5 illustrates the first surface 108 of the outer shell 102 with the inner layer 106 in the non-molded state. Alternatively, FIG. 6 illustrates a front view of the mouth guard in the molded state in accordance with the first embodiment. As shown, the inner layer 106 is folded up to conform to or otherwise mold to the wearer's teeth.

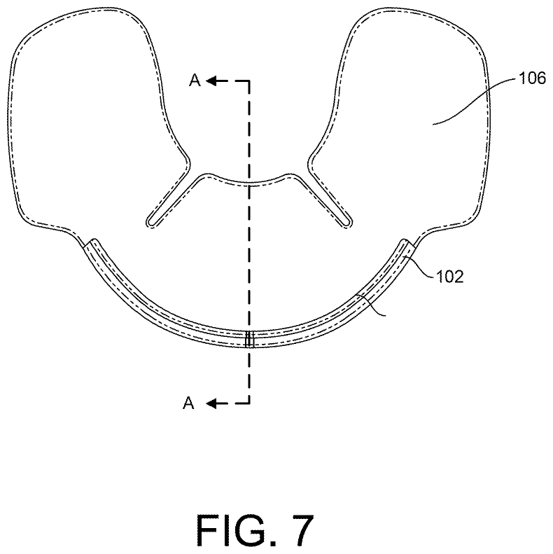

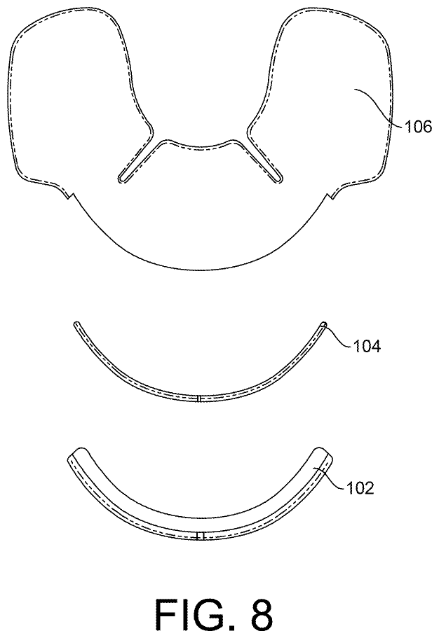

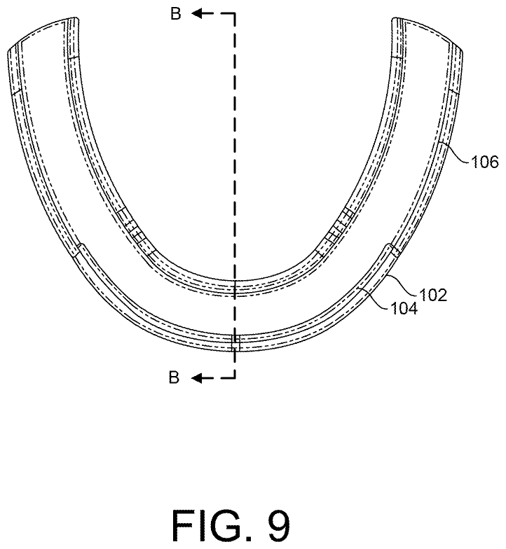

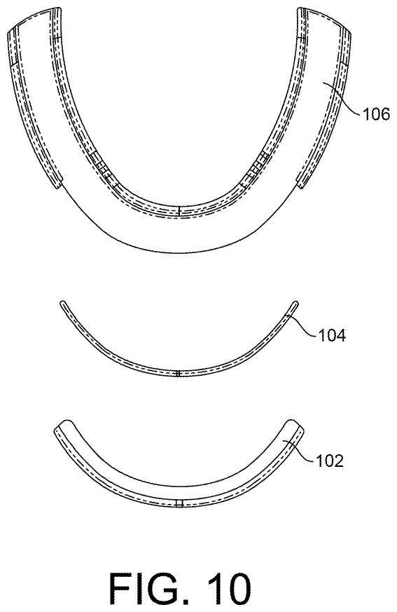

For further understanding, FIG. 7 illustrates a top view of the mouth guard in the non-molded state in accordance with the first embodiment, while FIG. 8 illustrates an exploded view from the top of the mouth guard in the non-molded state. Alternatively, FIG. 9 illustrates a top view of the mouth guard in the molded state, while FIG. 10 illustrates an exploded view from the top of the mouth guard in the molded state. As shown throughout the figures, the outer shell 102, the intermediate layer 104 and the inner layer 106 are molded together to form the mouth guard.

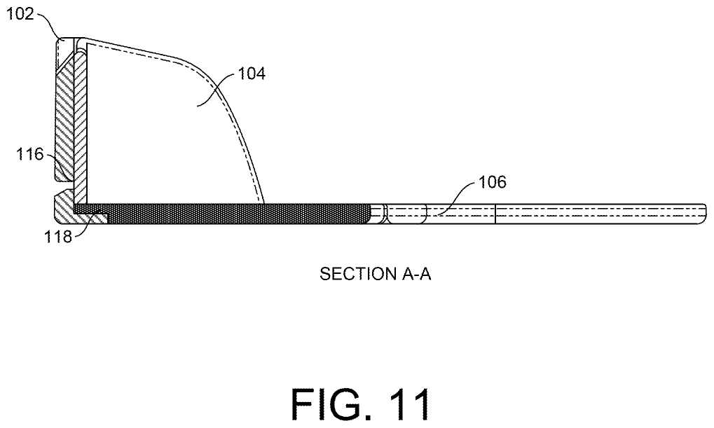

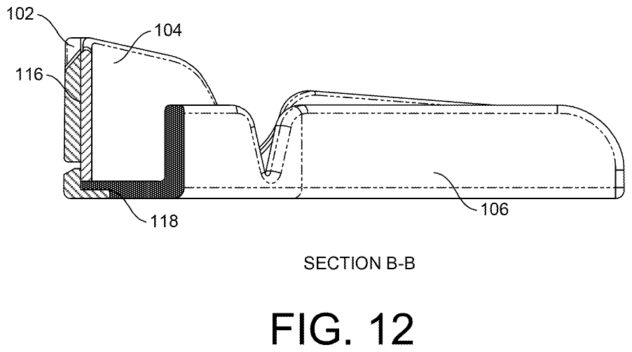

The adhesion or molding of the layers together can be seen clearly in FIGS. 11 and 12.

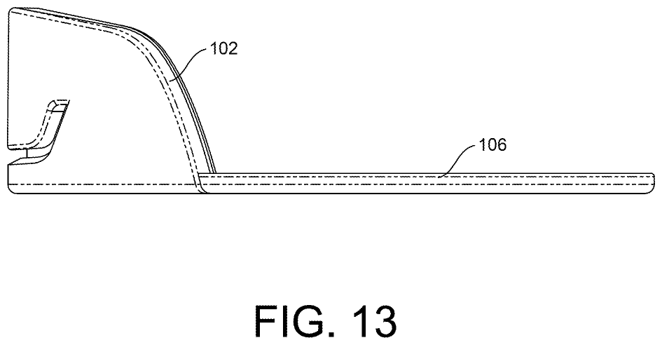

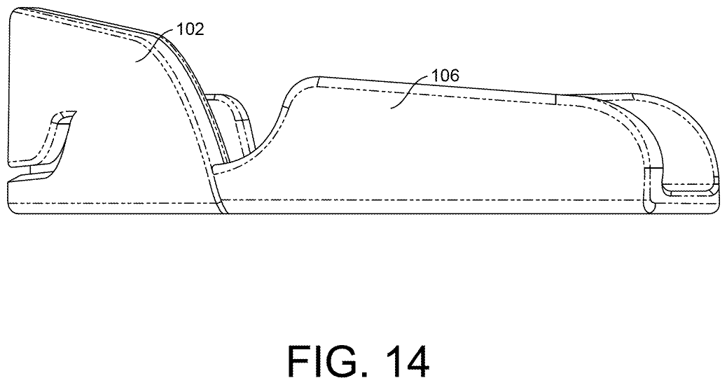

FIG. 11 illustrates a cross-sectional view of the mouth guard, taken from line A-A of FIG. 7, while FIG. 12 provides a cross-sectional view taken from line B-B of FIG. 9. The cross-sectional view illustrates example points of attachment (116 and 118) between the outer shell 102 and intermediate layer 104 (point 116) and between the inner layer 106 and outer shell 102 (point 118). Finally, FIG. 13 provides a side view of the mouth guard in the non-molded state, while FIG. 14 illustrates a side view of the mouth guard in the molded state. The unique design offers protection and enhanced fit in a low-profile form.

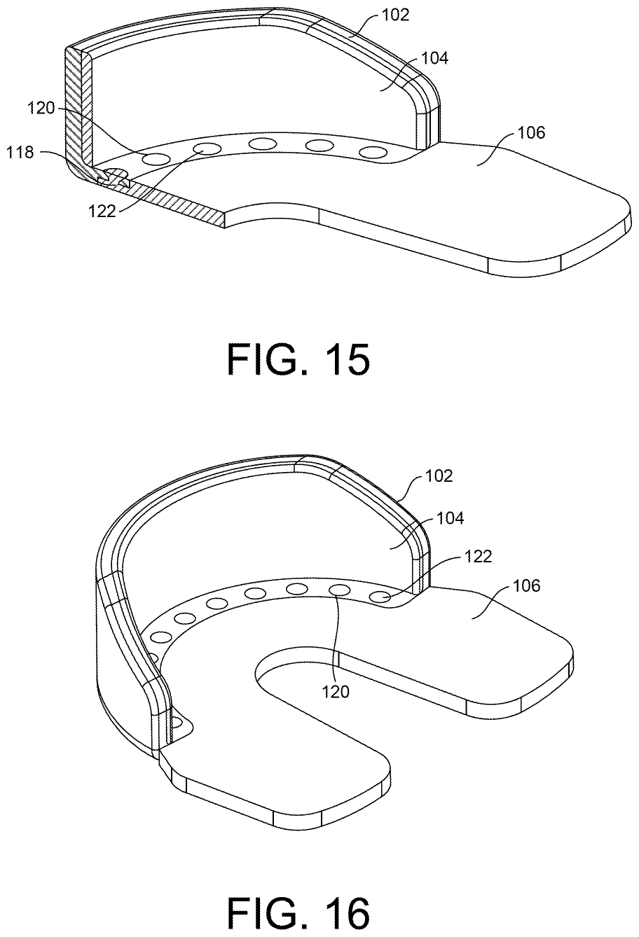

As noted above, the present disclosure provides improvements over the prior art through multiple unique embodiments. FIG. 15 through 24 illustrate a second embodiment that includes a unique mechanical interlocking mechanism. Specifically, FIG. 15 illustrates a cross-sectional view of an intermediate layer 104 over-molded onto an outer shell 102 of the mouth guard in accordance with the second embodiment. The inner layer 106 is also shown as connected to the outer shell 102. As can be appreciated by those skilled in the art, there are some challenge in blending of three layers formed of three distinct materials. Notably, the selected intermediate layer 104 and the outer shell 102 are easy to blend to form an attachment 116 via molding. However, in some aspects, adhering the inner layer 106 with the outer shell 102 imposes a limitation for molding with a desired material dataset. So, in this aspect, the inner layer 106 and the outer shell 102 are connected through mechanical interlocking 118. In this aspect, little minute geometries are included at the interface of the materials to provide mechanical attachment or the mechanical interlocking 118 arrangement. For example, small studs 122 can be formed that project from the inner layer 106, while countersunk holes can be formed in the outer shell 102 to receive the studs 122. Thus, when attached, the studs 122 pass through (or are formed through) the holes and flare into the countersunk holes 120.

In one aspect, the outer shell 102 is first molded from TPU or SBC since the outer shell 102 has the highest molding temperature and also has the highest shore hardness. Next, the intermediate layer 104 is over-molded onto the outer shell 102. Finally, the inner layer 106, which has the lowest molding temperature, is molded onto the outer shell 102. The small studs 122 fills the countersunk holes 120 in the outer shell 102, providing a strong mechanical interlock 118 to assist in the structural integrity as bonding of these materials together is a challenge. The higher level of hardness of the TPU (or SBC) substrate allows for “shut-offs” in the mold during the two over-molding steps to prevent flashing of the over-molded materials at the junctions of the materials.

For further understanding, FIG. 16 illustrates a perspective view of the mouth guard in accordance with the second embodiment. The mouth guard includes the outer shell 102 of non-moldable material, the intermediate 1004 and the inner layer 106. As noted above, the inner layer is of a non-moldable material (TPU or SBC or rubber), including a plurality of countersunk holes 120. The intermediate layer is of the first moldable material (EVA, or other selected material). The intermediate layer 104 is fixed to the second surface of the outer shell 102. The inner layer 106 is formed of a second moldable material (PCL) and includes a plurality of studs 122 that are received by the plurality of holes 120 to form the mouth guard 100.

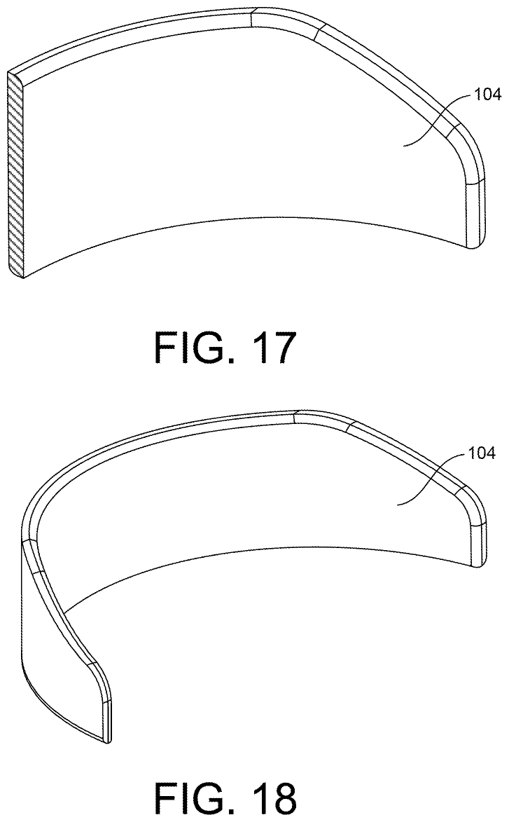

FIG. 17 illustrates a cross sectional view of the intermediate layer 104 of the mouth guard in accordance with the second embodiment, while FIG. 18 illustrates a perspective view of the intermediate layer 104.

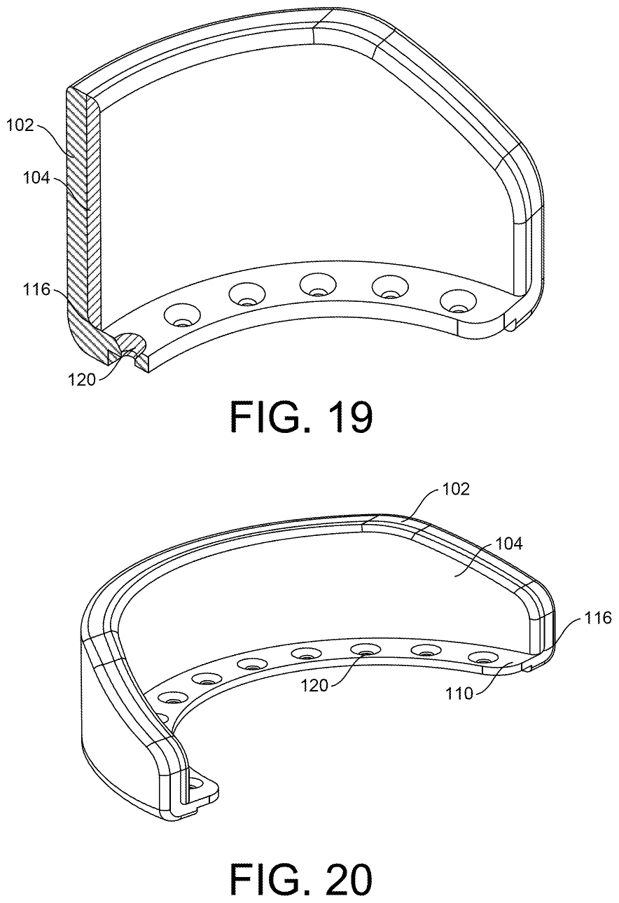

Further, FIGS. 19 and 20 illustrate a cross-sectional view and perspective view, respectively, of the intermediate layer 104 over-molded onto the outer shell 102. The intermediate layer 104 is an arc shaped layer which includes a front side and a back side. The front side of the intermediate layer 104 is attached or affixed to the outer shell 102, forming an attachment 116, and the backside faces the teeth of the user. The intermediate layer 106 is made of polyethylene polyvinyl acetate (EVA) material and the like and provides the customizable fitting of the mouth guard with the teeth. The intermediate layer 104 forms the attachment with the outer shell 102, leaving the holes 120 of the outer shell 102 exposed.

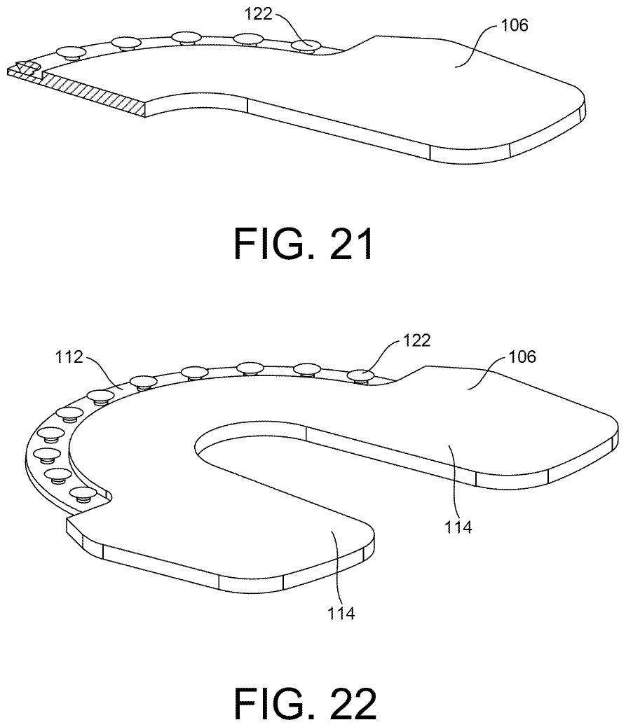

FIG. 21 illustrates a cross-sectional view of the inner layer 106 of the mouth guard in accordance with the second embodiment. As illustrated, the inner layer 106 is a horse-shoe shaped layer with a plurality of studs 122 to provide mechanical interlocking with the outer shell 102. Further, FIG. 22 illustrates a perspective view of the inner layer 106 depicting that the inner layer 106 is a horse shoe shaped layer with the pair of fins 114 and the arc 112 with a plurality of studs 122.

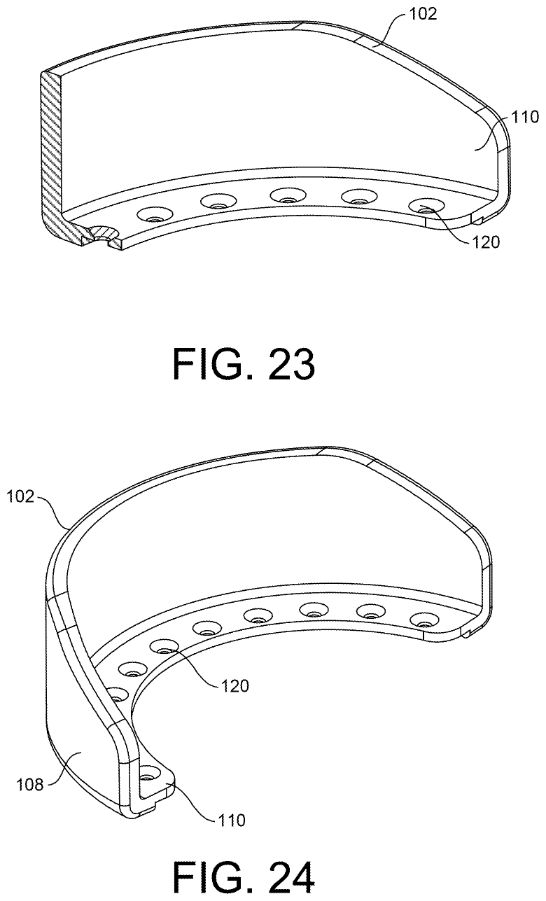

FIGS. 23 and 24 illustrate a cross-sectional view and perspective view, respectively, of the outer shell 102 of the mouth guard in accordance with the second embodiment. The outer shell 102 includes two surfaces, where the bottom periphery of the second surface 110 includes the plurality of holes 120 for engagement with the studs.

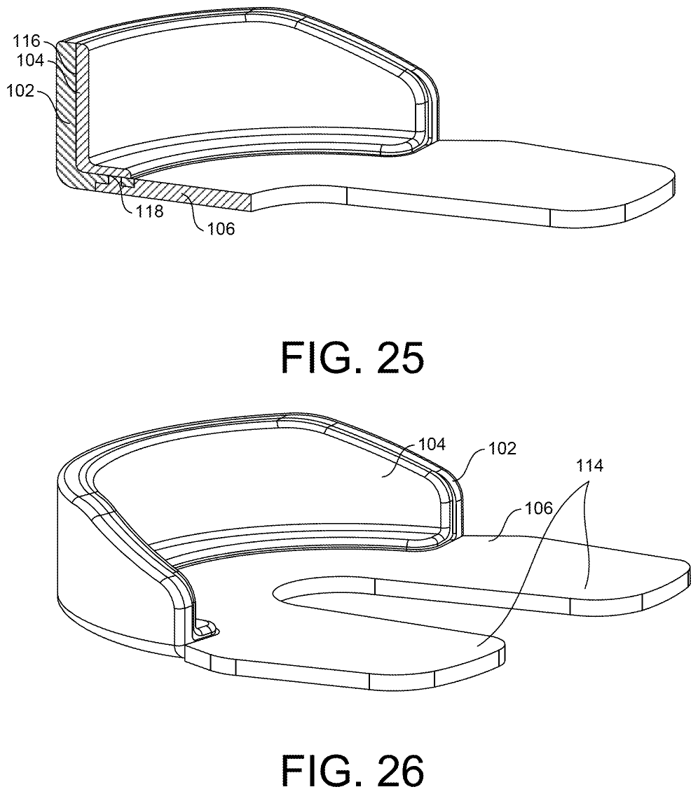

As noted above and illustrated in FIGS. 25 through 34, the present disclosure also provides a third embodiment of the mouth guard. Specifically, FIG. 25 illustrates a cross-sectional view of an intermediate layer 104 and inner layer 106 over-molded onto an outer shell 102 of the mouth guard in accordance with the third embodiment. The outer shell 102 is first molded from TPU or SBC. The outer shell 102 has the highest molding temperature and also has the highest shore hardness. Next the intermediate layer 104 (formed of EVA or other suitable material) is over-molded onto the outer shell 102, forming the first attachment 116. Finally, the inner layer 106 (formed from PCL), which has the lowest molding temperature, is molded onto the outer shell 102, thereby forming the second attachment 118. The mechanical interlocking 118 between the outer shell 102 and the intermediate layer 104 fills the holes formed in the outer shell 102.

As was the case with the second embodiment, the mechanical interlocking 118 assists in the structural integrity as bonding of these materials together is a challenge. The higher level of hardness of the TPU (or SBC) substrate allows for “shut-offs” in the mold during the two over-molding steps to prevent flashing of the over-molded materials at the junctions of the materials. The intermediate layer 104 covers the mechanical interlock features. This is partly for cosmetic reasons and also to ensure that none of the PCL posts can break off in a user's mouth. To be noted, in this aspect, the holes in the TPU (or SBC) substrate are not countersunk so that filling in of the holes during the over-molding of EVA can be prevented (no undercuts in the substrate tooling) so that these holes will later be filled with PCL in the final molding step. Note also that there is a shut-off provision (a small shoulder) on the PCL for the over-molding of the EVA and PCL.

FIG. 26 illustrate a perspective view of the intermediate layer 104 and the inner layer 106 over-molded onto the outer shell 102 of the mouth guard in accordance with the third embodiment. In this aspect, the outer shell 102 is first molded from TPU or SBC (or other suitable material). The outer shell 102 has the highest molding temperature and also has the highest shore hardness. Next, the intermediate layer 104 (formed from EVA or other suitable material) is over-molded onto the outer shell 102, thereby forming the first attachment 116. Finally, the inner layer 106 (formed from PCL or other suitable material), which has the lowest molding temperature, is molded onto the outer shell 102 to form the second attachment 118. As was the case above, the inner layer 106 includes a pair of fins 114 to easily fix to the palate.

The mechanical interlocking 118 between the outer shell 102 and the intermediate layer 104 fills the holes formed through the outer shell 102. The mechanical interlocking 118 assists in the structural integrity as bonding of these materials together is a challenge. The higher level of hardness of the TPU (or SBC) substrate allows for “shut-offs” in the mold during the two over-molding steps to prevent flashing of the over-molded materials at the junctions of the materials.

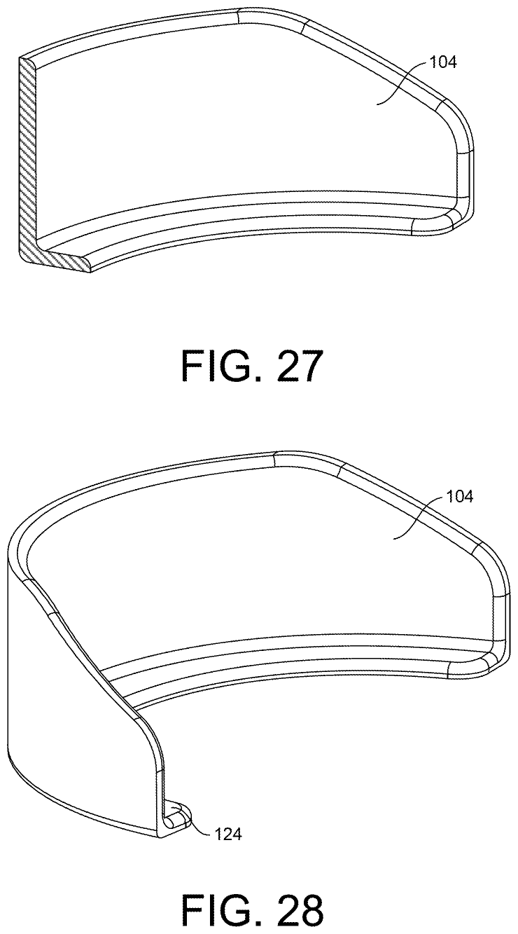

FIG. 27 illustrates a cross-sectional view of the intermediate layer 104 in accordance with the third embodiment. The intermediate layer 104 is made from the EVA over-mold. Further, FIG. 28 illustrates a perspective view of the intermediate layer 104 in accordance with the third embodiment, depicting that the intermediate layer 104 is formed as an arc shaped layer made from the EVA over-mold. Also shown is a bottom lip 124 that projects inward and is formed at the bottom of the intermediate layer 104. The bottom lip 124 covers the holes formed through the outer shell.

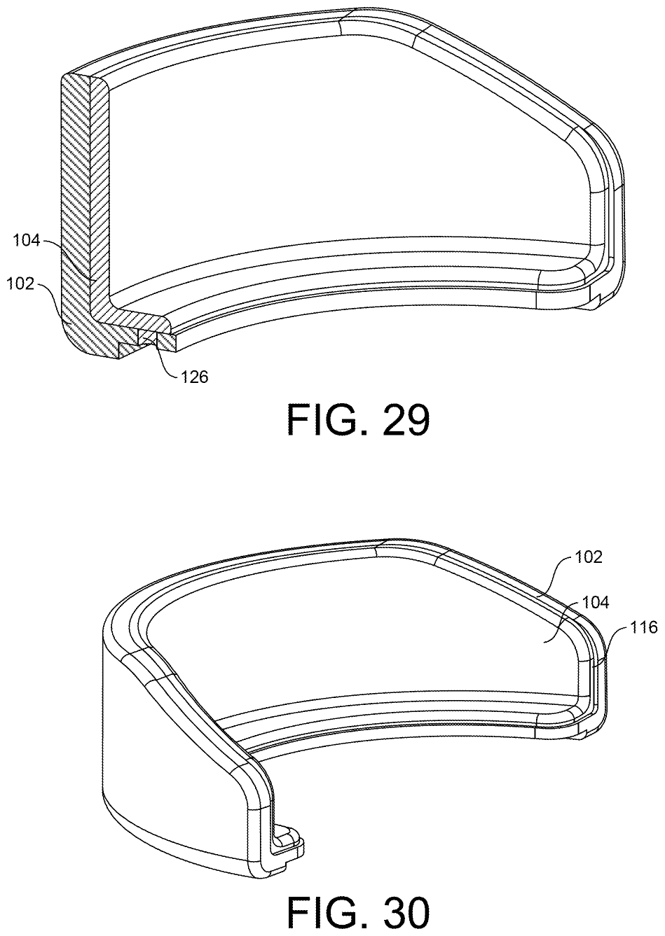

FIGS. 29 and 30 illustrate a cross sectional view and perspective view, respectively, of the intermediate layer 104 over-molded onto the outer shell 102 of the mouth guard in accordance with the third embodiment. The outer shell 102 is first molded from TPU or SBC (or other suitable material). The intermediate layer 104 is formed from EVA (or other suitable material) and is over-molded onto the outer shell 102, thereby forming first attachment 116. The intermediate layer 104 covers the mechanical interlock and forms a third attachment 126.

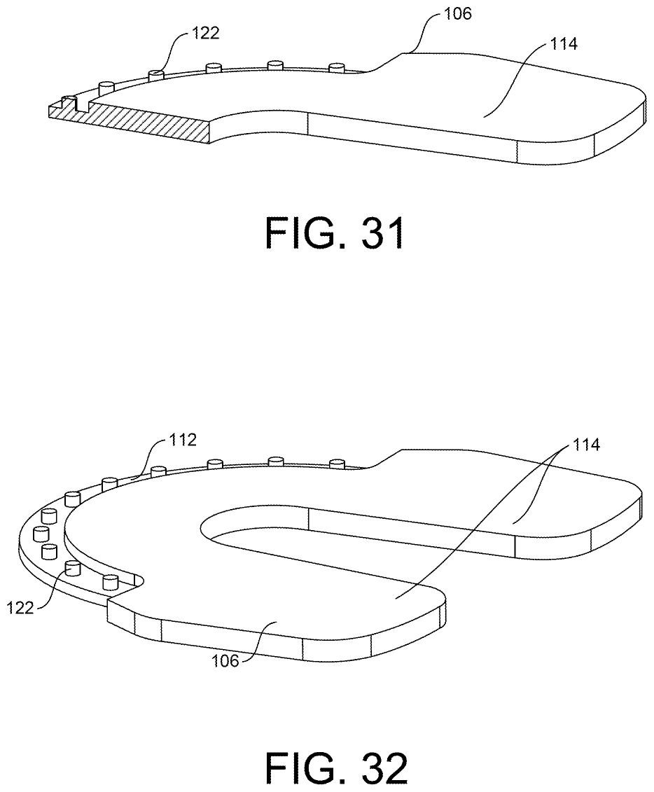

FIGS. 31 and 32 illustrates a cross-sectional view and perspective view, respectively, of the inner layer 106 in accordance with the third embodiment. The inner layer 106 is formed as a horse-shaped layer with a pair of fins 114. Further, the inner layer 106 includes an outward arc 112 that is formed with a plurality of studs 122 to provide mechanical interlocking with the outer shell 102

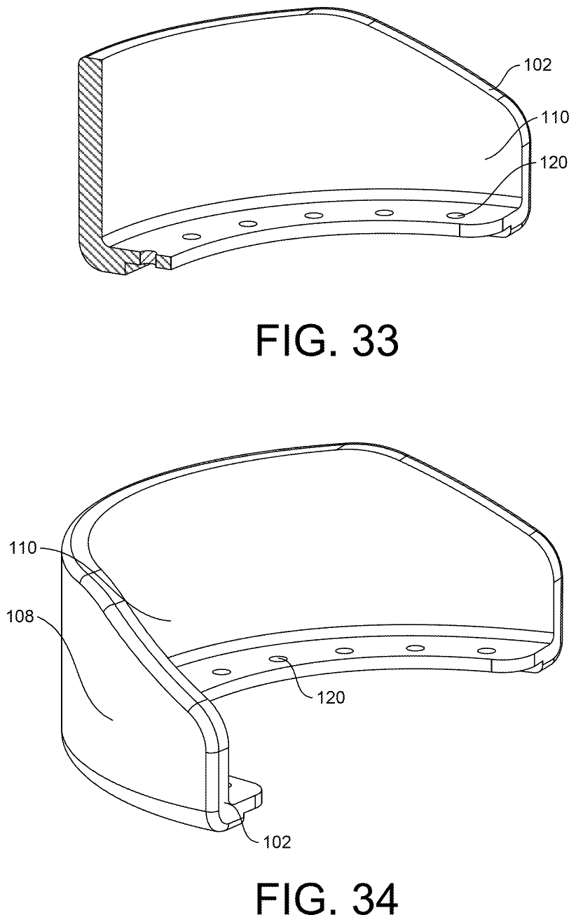

For further understanding, FIGS. 33 and 34 illustrate a cross-sectional and perspective view, respectively, of the outer shell in accordance with the third embodiment of the present invention. The outer shell 102 includes two surfaces, a first surface 108 facing the front and the second surface 110. The bottom periphery of the second surface 110 includes a plurality of holes 120 for the engagement with the plurality of studs.

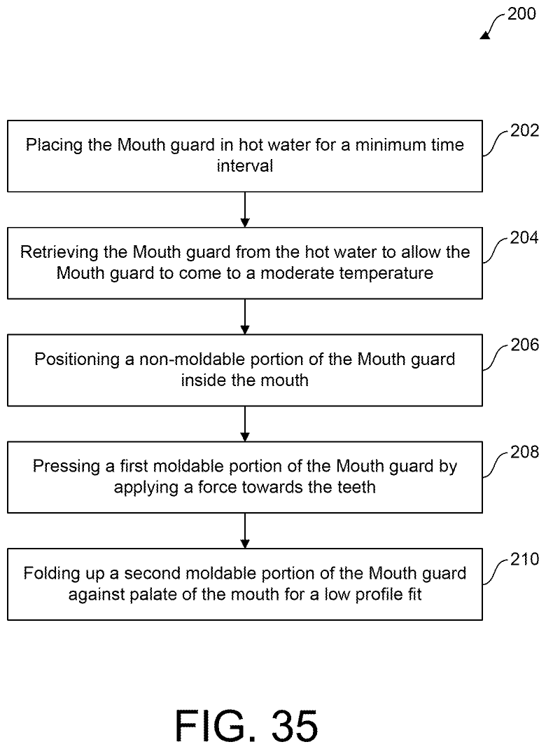

As noted above, the mouth guard can be shipped and sold in a low-profile form and thereafter molded by the user for a customized fit. A non-limiting example of the process is depicted in FIG. 35. Specifically, FIG. 35 illustrates a number of steps 200 relating to a method of placing the mouth guard 100 in the mouth of a user in accordance with an embodiment of the present invention. In the first step 202, the mouth guard is placed in hot water for a minimum time interval. This is followed by step 204 of retrieving the mouth guard 100 from the hot water. The mouth guard 100 is then allowed to come to a moderate temperature. Once the mouth guard 100 is at the moderate or room temperature, step 206 is executed to position a non-moldable portion of the mouth guard 100 inside the mouth. Thereafter, step 208 is performed by pressing the first moldable portion of the mouth guard 100 by applying a force towards the teeth. The final step 210 is then performed, which includes folding the second moldable portion of the mouth guard 100 against palate of the mouth for a low-profile fit.

While the various embodiments of the present invention have been described above, it should be understood that they have been presented by way of example only, and not of limitation. Likewise, the figure may depict an example architectural or other configuration for the invention, which is done to aid in understanding the features and functionality that can be included in the invention. The invention is not restricted to the illustrated example architectures or configurations, but the desired features can be implemented using a variety of alternative architecture and configurations.

Although, the invention is described above in terms of various exemplary embodiments and implementations, it should be understood that the various features, aspects, and functionality described in one or more of the individual embodiments are not limited in their applicability to the particular embodiment with which they are described, but instead can be applied, alone or in various combinations, to one or more of the other embodiments of the invention, whether or not such embodiments are described and whether or not such features are presented as being a part of a described embodiment. Thus, the breadth and scope of the present invention should not be limited by any of the above-described exemplary embodiments. Further, The presence of broadening words and phrases such as “one or more,” “at least,” “but not limited to” or other like phrases in some instances shall not be read to mean that the narrower case is intended or required in instances where such broadening phrases may be absent.

What is claimed is:

1. A mouth guard, comprising:
an outer shell formed of a non-moldable material, the outer shell including a first surface and a second surface opposite the first surface;
an intermediate layer formed of a first moldable material, wherein the intermediate layer is over-molded to the second surface of the outer shell; and
an inner layer formed of a second moldable material, wherein the inner layer is over-molded onto at least one of the intermediate layer and outer shell, and wherein the inner layer is transitionable from a first state to a second state, the first state being a substantially flat unmolded state and the second state being a molded state.

2. The mouth guard in accordance with claim 1, wherein the second surface of the outer shell includes a bottom periphery, the bottom periphery being formed with a plurality of holes passing therethrough.

3. The mouth guard in accordance with claim 2, wherein the inner layer includes a plurality of posts, the plurality of posts being formed through the plurality of holes.

4. The mouth guard in accordance with claim 3, wherein the intermediate layer is formed to cover the plurality of hole and posts formed therethrough.

5. The mouth guard in accordance with claim 4, wherein the plurality of holes are formed as countersunk holes, with the plurality of posts flaring into the countersunk holes.

6. The mouth guard in accordance with claim 5, wherein the outer shell is made up of a material selected from a group consisting of Thermoplastic urethane, Styrene Block Polymer or rubber,

7. The mouth guard in accordance with claim 6, wherein the inner layer is made of Polycaprolactate (PCL) material.

8. The mouth guard in accordance with claim 7, wherein the intermediate layer is made of polyethylene polyvinyl acetate (EVA) material.

9. The mouth guard in accordance with claim 1, wherein the outer shell is made up of a material selected from a group consisting of Thermoplastic urethane, Styrene Block Polymer or rubber,

10. The mouth guard in accordance with claim 1, wherein the inner layer is made of Polycaprolactate (PCL) material.

11. The mouth guard in accordance with claim 1, wherein the intermediate layer is made of polyethylene polyvinyl acetate (EVA) material.

12. A method for placing a mouth guard in the mouth of a user for a low profile fit, the method comprising acts of:
placing the mouth guard in hot water for a minimum time interval;
retrieving the mouth guard from the hot water to allow the mouth guard to come to a moderate temperature;
positioning a non-moldable portion of the mouth guard inside the mouth;
pressing a first moldable portion of the mouth guard by applying a force towards the teeth; and
folding up a second moldable portion of the mouth guard against palate of the mouth for a low profile fit.

13. The method for placing the mouth guard in accordance with claim 12, wherein the first moldable portion is made of polyethylene polyvinyl acetate (EVA) material.

14. The method for placing the mouth guard in accordance with claim 12, wherein the second moldable portion is made of Polycaprolactate (PCL) material.

15. The method for placing the mouth guard in accordance with claim 12, wherein the non-moldable portion is selected from thermoplastic Urethane, Styrene Block Polymer or rubber.
